(19) United States
(12) Patent Application Publication
(10) Pub. No.: US 2025/0111281 A1
(43) Pub. Date: Apr. 3, 2025

(54) CONTRASTIVE LEARNING FOR IMPLICIT NEURAL REPRESENTATIONS

(71) Applicant: Elekta Limited, Crawley (GB)

(72) Inventors: Samuele PAPA, Crawley (GB); Riccardo VALPERGA, Crawley (GB); Jan-Jakob Sonke, Amsterdam (NL); Efstratios GAVVES, Crawley (GB)

(21) Appl. No.: 18/899,744

(22) Filed: Sep. 27, 2024

(30) Foreign Application Priority Data
Sep. 29, 2023 (GB) 2314992.5

(51) Int. Cl. G06N 20/00; G06V 10/74; G06V 10/762; G06V 10/764; G16H 30/40

(52) U.S. Cl. CPC G06N 20/00; G06V 10/761; G06V 10/762; G06V 10/764; G16H 30/40

(57) ABSTRACT

A method for training an ML model to generate lower dimensional representations of a plurality of Implicit Neural Representations (INRs) can include obtaining a plurality of INRs, each INR representing a medical image. The method can further comprise, for each of the plurality of INRs, repeating, at least twice, identifying an augmentation for application to the INR, applying the identified augmentation to the INR, and inputting the augmented version of the INR to an encoder ML model. The encoder ML model can output a vector representation of the augmented version of the INR. The vector representation is of a lower dimension than the INR. The method can further comprise determining a pairwise similarity between pairs of vector representations output by the encoder ML model and updating trainable parameters of the encoder ML model to minimize a loss function. The loss function rewards increased similarity between vector representations of augmented versions of the same INR.

### TECHNICAL FIELD

The present disclosure relates to a computer implemented method for training a Machine Learning (ML) model to generate lower dimensional representations of a plurality of Implicit Neural Representations (INR), each INR representing a medical image. The present disclosure also relates to a processing node, and to a computer program product configured, when run on a computer, to carry out methods for training an ML model to generate lower dimensional representations of INRs.

### BACKGROUND REFERENCES

### References

Samuel K Ainsworth, Jonathan Hayase, and Siddhartha Srinivasa. Git re-basin: Merging models modulo permutation symmetries. arXiv preprint arXiv:2209.04836, 2022.
Suzanna Becker and Geoffrey Hinton. Self-organizing neural network that discovers surfaces in random-dot stereograms. Nature, 1992.
An Mei Chen. Haw-minn Lu, and Robert Hecht-Nielsen. On the geometry of feedforward neural network error surfaces. Neural computation, 5(6):910-927, 1993.
Ting Chen, Simon Kornblith, Mohammad Norouzi, and Geoffrey Hinton. A simple framework for contrastive learning of visual representations. In International conference on machine learning, pp. 1597-1607. PMLR, 2020.
Gabriele Corso, Luca Cavalleri, Dominique Beaini, Pietro Lio', and Petar Velic ̌kovic'. Principal neighbourhood aggregation for graph nets. In Advances in Neural Information Processing Systems (NeurIPS), 2020.
Terrance DeVries and Graham W Taylor. Improved regularization of convolutional neural networks with cutout. arXiv preprint arXiv:1708.04552, 2017.
Cameron Diao and Ricky Loynd. Relational attention: Generalizing transformers for graph-structured tasks. In International Conference on Learning Representations (ICLR), 2023.
Emilien Dupont, Hyunjik Kim, S M Eslami, Danilo Rezende, and Dan Rosenbaum. From data to functa: Your data point is a function and you can treat it like one. arXiv preprint arXiv:2201.12204, 2022.
Gabriel Eilertsen, Daniel Jo\ddot{ }nsson, Timo Ropinski, Jonas Unger, and Anders Ynnerman. Classifying the classifier dissecting the weight space of neural networks. arXiv preprint arXiv:2002.05688, 2020.
Rahim Entezari, Hanie Sedghi, Olga Saukh, and Behnam Neyshabur. The role of permutation invariance in linear mode connectivity of neural networks. arXiv preprint arXiv:2110.06296, 2021.
Rizal Fathony, Anit Kumar Sahu, Devin Willmott, and J Zico Kolter. Multiplicative filter networks. In International Conference on Learning Representations, 2020.
Spyros Gidais, Praveer Singh, and Nikos Komodakis. Unsupervised representation learning by predicting image rotations. arXiv preprint arXiv:1803.07728, 2018.
Jean-Bastien Grill, Florian Strub, Florent Aftche', Corentin Tallec, Pierre Richemond, Elena Buchatskaya, Carl Doersch, Bernardo Avila Pires, Zhaohan Guo, Mohammad Gheshlaghi Azar, et al. Bootstrap your own latent-a new approach to self-supervised learning. Advances in neural information processing systems, 33:21271-21284, 2020.
Kaiming He, Haoqi Fan, Yuxin Wu, Saining Xie, and Ross Girshick. Momentum contrast for unsupervised visual representation learning. In Proceedings of the IEEE/CVF conference on computer vision and pattern recognition, pp. 9729-9738, 2020.
Robert Hecht-Nielsen. On the algebraic structure of feedforward network weight spaces. In Advanced Neural Computers, pp. 129-135. Elsevier, 1990.
Andrew G Howard. Some improvements on deep convolutional neural network based image classification. arXiv preprint arXiv:1312.5402, 2013.
Ashish Jaiswal, Ashwin Ramesh Babu, Mohammad Zaki Zadeh, Debapriya Banerjee, and Fillia Makedon. A survey on contrastive self-supervised learning. Technologies, 9(1):2, 2020.
Diederik P Kingma and Jimmy Ba. Adam: A method for stochastic optimization. arXiv preprint arXiv:1412.6980, 2014.
Phuc H Le-Khac, Graham Healy, and Alan F Smeaton. Contrastive representation learning: A framework and review. Ieee Access, 8:193907-193934, 2020.
Tianye Li, Mira Slavcheva, Michael Zollhoefer, Simon Green, Christoph Lassner, Changil Kim, Tanner Schmidt, Steven Lovegrove, Michael Goesele, Richard Newcombe, et al. Neural 3d video synthesis from multi-view video. In Proceedings of the IEEE/CVF Conference on Computer Vision and Pattern Recognition, pp. 5521-5531, 2022.
Ben Mildenhall, Pratul P Srinivasan, Matthew Tancik, Jonathan T Barron, Ravi Ramamoorthi, and Ren Ng. Nerf: Representing scenes as neural radiance fields for view synthesis. Communications of the ACM, 65(1):99-106, 2021.
Aviv Navon, Aviv Shamsian, Idan Achituve, Ethan Fetaya, Gal Chechik, and Haggai Maron. Equivariant architectures for learning in deep weight spaces. arXiv preprint arXiv:2301.12780, 2023.
Jeong Joon Park, Peter Florence, Julian Straub, Richard Newcombe, and Steven Lovegrove. Deepsdf: Learning continuous signed distance functions for shape representation. In Proceedings of the IEEE/CVF conference on computer vision and pattern recognition, pp. 165-174, 2019.
Albert Pumarola, Enric Corona, Gerard Pons-Moll, and Francesc Moreno-Noguer. D-nerf-Neural radiance fields for dynamic scenes. In Proceedings of the IEEE/CVF Conference on Computer Vision and Pattern Recognition, pp. 10318-10327, 2021.
Konstantin Schu\ddot{ }rholt, Dimche Kostadinov, and Damian Borth. Self-supervised representation learning on neural network weights for model characteristic prediction. Advances in Neural Information Processing Systems, 34:16481-16493, 2021.
Berfin Simsek, Franc ̧ois Ged, Arthur Jacot, Francesco Spadaro, Cle'ment Hongler, Wulfram Gerstner, and Johanni Brea. Geometry of the loss landscape in overparameterized neural networks: Symmetries and invariances. In International Conference on Machine Learning, pp. 9722-9732. PMLR, 2021.
Vincent Sitzmann, Julien Martel, Alexander Bergman, David Lindell, and Gordon Wetzstein. Implicit neural representations with periodic activation functions. Advances in neural information processing systems, 33:7462-7473, 2020.
Christian Szegedy, Wei Liu, Yangqing Jia, Pierre Sermanet, Scott Reed, Dragomir Anguelov, Dumitru Erhan, Vincent Vanhoucke, and Andrew Rabinovich. Going deeper with convolutions. In Proceedings of the IEEE conference on computer vision and pattern recognition, pp. 1-9, 2015.
Matthew Tancik, Pratul Srinivasan, Ben Mildenhall, Sara Fridovich-Keil, Nithin Raghavan, Utkarsh Singhal, Ravi Ramamoorthi, Jonathan Barron, and Ren Ng. Fourier features let networks learn high frequency functions in low dimensional domains. Advances in Neural Information Processing Systems, 33:7537-7547, 2020.
Matthew Tancik, Ben Mildenhall, Terrance Wang, Divi Schmidt, Pratul P Srinivasan, Jonathan T Barron, and Ren Ng. Learned initializations for optimizing coordinate-based neural representations. In Proceedings of the IEEE/CVF Conference on Computer Vision and Pattern Recognition, pp. 2846-2855, 2021.
Thomas Unterthiner, Daniel Keysers, Sylvain Gelly, Olivier Bousquet, and Ilya Tolstikhin. Predicting neural network accuracy from weights. arXiv preprint arXiv:2002.11448, 2020.
Laurens Van der Maaten and Geoffrey Hinton. Visualizing data using t-sne. Journal of machine learning research, 9(11), 2008.
Yiheng Xie, Towaki Takikawa, Shunsuke Saito, Or Litany, Shiqin Yan, Numair Khan, Federico Tombari, James Tompkin, Vincent Sitzmann, and Srinath Sridhar. Neural fields in visual computing and beyond. In Computer Graphics Forum, volume 41, pp. 641-676. Wiley Online Library, 2022.
Dejia Xu, Peihao Wang, Yifan Jiang, Zhiwen Fan, and Zhangyang Wang. Signal processing for implicit neural representations. Advances in Neural Information Processing Systems, 35:13404-13418, 2022.
Yuan Yin, Matthieu Kirchmeyer, Jean-Yves Franceschi, Alain Rakotomamonjy, and Patrick Gallinari. Continuous pde dynamics forecasting with implicit neural representations. arXiv preprint arXiv:2209.14855, 2022.
David W Zhang, Miltiadis Kofinas, Yan Zhang, Yunlu Chen, Gertjan J Burghouts, and Cees G M Snoek. Neural networks are graphs! graph neural networks for equivariant processing of neural networks. 2023.
Allan Zhou, Kaien Yang, Kaylee Burns, Yiding Jiang, Samuel Sokota, J Zico Kolter, and Chelsea Finn. Permutation equivariant neural functionals. arXiv preprint arXiv:2302.14040, 2023

### SUMMARY

Implicit Neural Representations (INR) have emerged as an alternative to traditional array representations of data. For example, color images are commonly processed as grids of RGB pixel intensities, and 3D shapes are commonly processed as voxel occupancies. In contrast to this traditional approach, INRs are functions that map, in the case of color images, coordinates to RGB values. In this manner, an INR respects the continuous nature of the underlying signal to be represented, as opposed to discretizing, via a grid, what is often a continuous underlying signal.

In general, INRs can be used with a range of data modalities, in particular, they have gained traction in computer vision to represent not only images, but also videos, 3D shapes and scenes, medical images and audio. The benefits of such functional representations include the ability to process the data at an arbitrary resolution, and to deal with data that is difficult to discretize such as signals that are sampled irregularly on grids with inconsistent shapes across the dataset. Even signals on non-Euclidian manifolds, such as complex PDE solutions and climate data, can be handled more naturally with INRs. Furthermore, INRs allow for more memory/compute efficient training for downstream tasks since ideally, the dimension of the INR can be significantly lower than that of the array representing the signal.

Considering the example of medical images specifically, computed Tomography (CT) is a medical imaging technique for reconstructing material density inside a patient, using the mathematical and physical properties of X-ray scanners. In CT, several X-ray scans, or projections, of the patient are acquired from various angles using a detector, and reconstruction methods are then used to create a three-dimensional image of the patient volume from the two-dimensional measurement data in the projections. An important variant of CT is Cone Beam CT (CBCT), which uses flat panel detectors to scan a large fraction of the volume in a single rotation. CBCT reconstruction is more difficult than reconstruction for classical (helical) CT, owing to the inherent mathematical difficulty of Radon Transform inversion in the three-dimensional setting, physical limits of the detector, and characteristics of the measurement process such as noise.

INRs may offer particular advantages for the representation of medical images such as CT and CBCT images, owing to their ability to process data at an arbitrary resolution. For example, CT scans may result in different resolutions across different axes. In a conventional setting, this may be addressed by interpolating between slices for the axes with the lower resolution. However, with an INR that can be sampled an any resolution, this issue is completely avoided.

Motivated by the advantages offered by INRs, recent works have shown promising results in performing deep learning tasks, such as classification and generation, directly on implicit representations. However, this new paradigm shift comes with significant challenges. Performing a deep learning task directly on an INR involves submitting the parameters of the INR, that is the weights and biases of the Neural Field that represents the respective underlying signal, to the relevant deep learning model. Deep learning models are generally adapted for inputs that lie on a low dimensional manifold. However, this is not the case for the parameters of a neural network, meaning many deep learning models can struggle with processing and interpreting INRs.

It is an aim of the present disclosure to provide a method, a processing node, and a computer program product which at least partially address one or more of the challenges mentioned above. It is a further aim of the present disclosure to provide a method, a processing node, and a computer program product which cooperate to enable the training of an ML model to generate of a lower dimensionality representation of an INR.

According to a first aspect of the present disclosure, there is provided a computer implemented method for training a Machine Learning (ML) model to generate lower dimensional representations of a plurality of Implicit Neural Representations (INRs) each INR representing a medical image. The method comprises obtaining a plurality of INRs, each INR representing a medical image. The method further comprises, for each of the plurality of INRs, repeating, at least twice, the steps of (i) identifying an augmentation for application to the INR, (ii) applying the identified augmentation to the INR, and (iii) inputting the augmented version of the INR to an encoder ML model, wherein the encoder ML model is operable to output a vector representation of the augmented version of the INR, and wherein the vector representation is of a lower dimension than the INR. The method further comprises determining a pairwise similarity between pairs of vector representations output by the encoder ML model, and updating trainable parameters of the encoder ML model to minimize a loss function, wherein the loss function rewards increased similarity between vector representations of augmented versions of the same INR.

According to another aspect of the present disclosure, there is provided a computer implemented method for generating lower dimensional representation of an INR of a medical image, wherein the representation is for use in an ML task. The method comprises obtaining an INR representing a medical image, and inputting the INR to an encoder ML model. According to the method, the encoder ML model is operable to output a vector representation of the INR, the vector representation being of a lower dimension than the INR, and the encoder ML model has been trained using a method according to any aspect or example of the present disclosure.

According to another aspect of the present disclosure, there is provided a computer program product comprising a computer readable medium, the computer readable medium having computer readable code embodied therein, the computer readable code being configured such that, on execution by a suitable computer or processor, the computer or processor is caused to perform a method according to any one or more aspects or examples of the present disclosure.

According to another aspect of the present disclosure, there is provided a processing node for training an ML model to generate lower dimensional representations of a plurality of INRs, each INR representing a medical image. The processing node comprises processing circuitry configured to cause the processing node to obtain a plurality of INRs, each INR representing a medical image. The processing circuitry is further configured to cause the processing node to, for each of the plurality of INRs, repeat, at least twice, the steps of (i) identifying an augmentation for application to the INR, (ii) applying the identified augmentation to the INR, and (iii) inputting the augmented version of the INR to an encoder ML model, wherein the encoder ML model is operable to output a vector representation of the augmented version of the INR, and wherein the vector representation is of a lower dimension than the INR. The processing circuitry is further configured to cause the processing node to determine a pairwise similarity between pairs of vector representations output by the encoder ML model, and update trainable parameters of the encoder ML model to minimize a loss function, wherein the loss function rewards increased similarity between vector representations of augmented versions of the same INR.

According to another aspect of the present disclosure, there is provided radiotherapy treatment apparatus comprising a processing node according to any one of the aspects or examples of the present disclosure.

### BRIEF DESCRIPTION OF THE DRAWINGS

For a better understanding of the present disclosure, and to show more clearly how it may be carried into effect, reference will now be made, by way of example, to the following drawings in which:

FIG. 1 is a representation an example implementation of methods discussed herein, applied to publicly available datasets;

FIG. 2 illustrates aliasing in multiplicative filters networks;

FIG. 3 shows Table 1, which illustrates hyperparameters used in the datasets of Implicit Neural Representations;

FIG. 4 illustrates embeddings of the datasets using t-SNE;

FIG. 5 shows Table 2, which illustrates accuracy (%) for INRs used in experiments described herein;

FIG. 6 illustrates an example diagram of a method for training a Machine Learning (ML) model to generate lower dimensional representations of Implicit Neural Representations using medical images; and

FIG. 7 illustrates a block diagram of example of a processing node upon which one or more embodiments may be implemented.

### DETAILED DESCRIPTION

Examples of the present disclosure present methods and nodes that enable the training of an ML model to generate of a lower dimensionality representation of an INR.

As discussed above, Implicit Neural Representations (INRs) are functional representations of discretely sampled continuous signals. Namely, INRs are the parameters of neural network fields:

$${f}_{θ} : {ℝ}^{d} \to {ℝ}^{c} \qquad Equation 1$$

where the input dimension d is the signal domain dimension and c is the number of signal channels. For an RGB image, for instance, d=2, c=3, and its implicit representation are the parameters of a function f_{θ} fitted to map coordinates of a 2D grid to the corresponding RGB values of the image. Particularly, to obtain the INR of a discrete signal {Iᵢ}N at N discrete locations, we fit the parameters θ of f_{θ} to minimize the reconstruction loss:

$$ℒ ( θ , {\{ {I}_{i} \}}_{i = 1}^{N} ) = {\sum }_{i = 1}^{N} {\vert {f}_{θ} ( {x}_{i} ) - {I}_{i} \vert}_{2}^{2} \qquad Equation 2$$

where xᵢ are the coordinate locations on the domain of the signal. Unlike discrete representations, which rely on fixed-size arrays to contain data, implicit representations are a much more natural choice for continuous signals, offering a new paradigm for representing complex, high-dimensional data in a compact, efficient manner (Park et al., 2019: Mildenhall et al., 2021: Tancik et al., 2020; Xie et al., 2022; Yin et al., 2022; Pumarola et al., 2021; Li et al., 2022). However, one of the challenges that has gained substantial attention lies in performing downstream tasks directly on these implicit representations.

Prior to the exploration of implicit neural representations, the domain of self-supervised representation learning has shown impressive results in enabling features extraction without explicit supervision, i.e., from unlabeled data. Originally, this overcame the problem of the high cost of data annotation in supervised learning (Le-Khac et al., 2020). One of the most successful among the self-supervised learning frameworks is contrastive learning (Jaiswal et al., 2020). Despite its remarkable success in different domains and with downstream tasks, the use of self-supervised learning, and in particular contrastive learning, with implicit representations has remained unexplored. The main reason for this is that the operations required to perform contrastive learning on array representations of images are not straightforward to translate to their implicit representations. To the best of our knowledge, this work is the first that demonstrates the applicability of contrastive learning techniques to implicit neural representations, with the exception of Navon et al. (2023) that suggested this direction in a simplified setting with a dataset of sinusoidal. We think that this is an important step forward in the representation learning domain for the following reasons:

Neural fields are resolution independent. The same architecture can be trained to reconstruct images of different resolution and shape. One key challenge of self-supervised methods is learning representations that generalize well across datasets of different shapes and image sizes.

Some modalities, such as scenes, shapes and audio do not have array representations that can be easily adapted to work with existing self-supervised methods.

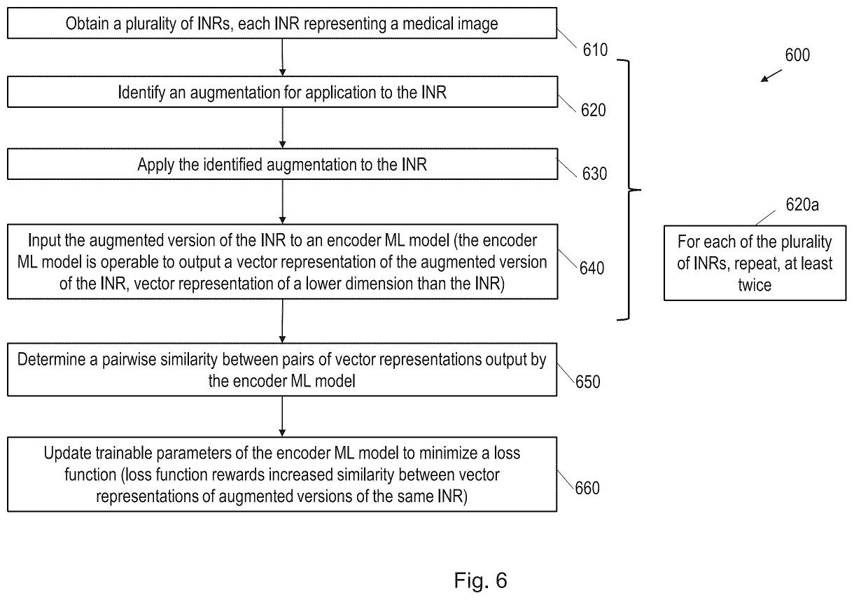

A first example computer implemented method (600) is presented herein and in FIG. 6. The method is for training a Machine Learning (ML) model to generate lower dimensional representations of a plurality of Implicit Neural Representations (INRs), wherein each INR represents a medical image. The method may be performed by a processing node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualized function, or any other logical entity. The processing node may encompass multiple logical entities, as discussed in greater detail below.

In a first step 610, the method comprises obtaining a plurality of INRs, each INR representing a medical image. The method then comprises, for each of the plurality of INRs, repeating, at least twice (as illustrated at 620a), the steps of (i) identifying an augmentation for application to the INR in step 620, (ii), applying the identified augmentation to the INR in step 630, and (iii) inputting the augmented version of the INR to an encoder ML model in step 640, wherein the encoder ML model is operable to output a vector representation of the augmented version of the INR, and wherein the vector representation is of a lower dimension than the INR. The method then comprises determining a pairwise similarity between pairs of vector representations output by the encoder ML model in step 650, and updating trainable parameters of the encoder ML model to minimize a loss function in step 660, wherein the loss function rewards increased similarity between vector representations of augmented versions of the same INR.

The disclosed method applies contrastive learning to learn how to generate a structured, lower dimensional representation of an INR. In this context “lower dimensional” refers to the representation being of a lower dimension than the INR itself. This lower dimensional representation preserves the essential information in the INR for representing the medical image, but is more suited to be input to existing ML models for performing downstream ML tasks on the representations. The method takes information in the weight space of a Neural Field, that is the weights and biases in individual INRs of medical images, and outputs a lower dimensional representation of the information, suitable for downstream ML tasks. In effect, the method learns to encode INRs such that regardless of the augmentation applied to a given INR, all augmented versions of the same INR will be clustered in the same region of the relevant latent space in which they are encoded. The augmentations are applied to the INRs, that is in the weight space, allowing for learning of an encoded representation of the INR itself, as opposed to the image it represents. In this manner, the advantages of using INRs for representing medical images and for downstream ML tasks are maintained, while addressing the issue of the high dimensionality of INRs when used as input to downstream ML tasks.

According to examples of the present disclosure, the method steps may be iterated until a convergence criterion is reached. The convergence criterion may be a function of any one or more of: an evolution of a value of the loss function, a number of iterations, a total training time, etc.

In some examples, the method can be implemented using the SimCLR framework, as discussed in greater detail below.

As discussed above, an INR, or Neural Field, is a neural architecture that parameterizes a field, i.e., a quantity defined over spatial and/or temporal coordinates, using a neural network. An INR may thus comprise, for example, the values of trained parameters of the neural network that parameterizes the field, including for example the weights and biases of the neural network. A neural network is an example of a Machine Learning (ML) model. Another example of an ML model is the encoder ML model referred to in the above disclosed method. For the purposes of the present disclosure, the term “ML model” encompasses within its scope the following concepts:
machine Learning algorithms, comprising processes or instructions through which data may be used in a training process to generate a model artefact for performing a given task, or for representing a real-world process or system; and
the model artefact that is created by such a training process, and which comprises the computational architecture that performs the task.

According to examples of the present disclosure, an augmentation comprises a change in the INR, that is a change to the weights and/or biases of the Neural Field that parameterizes the medical image. The augmentation may result in a change to the representation of the medical image that is provided by the INR. However, if a change to the represented image is caused, that change does not impact the overall information provided by the representation of the image, but may be envisaged for example as providing an alternative “view” of the image (for example, scaling, rotating, translating, or cropping the image). Thus, while the “surface appearance” of the represented image may be different, the cognitive content of the image is unchanged. By rewarding similarity of vector representations of the same INR after application of different augmentations, the encoder ML model learns to perform encoding that maintains the most important information within the INR in terms of its functionality in representing the medical image. That is, the encoder ML model learns to encode the INRs in such a manner that the most important information for parameterizing the medical image is maintained, while still reducing the dimensionality of the original INR, to facilitate processing by downstream ML tasks.

Another example computer implemented method is presented herein for training a Machine Learning (ML) model to generate lower dimensional representations of a plurality of Implicit Neural Representations (INRs), wherein each INR represents a medical image. As for the method 600 presented above, the method presented below may be performed by a processing node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualized function, or any other logical entity. The processing node may encompass multiple logical entities, as discussed in greater detail below. The method presented below illustrates an example of how the method presented above may be implemented and supplemented to achieve the above discussed and additional functionality.

In a first step, the method comprises obtaining a plurality of INRs, each INR representing a medical image. In some examples of the present disclosure, the INRs may comprise Multiplicative Filter Networks (MFNs). MFNs have the advantage that when applying an augmentation to the network such that the represented or output image from the network is rotated, translated, or scaled, the percentage of weights changed in an MFN is higher than for a majority of other Neural Field architectures presently in use. By changing a higher percentage of weights in the INR, it may be envisaged that a more robust training of the encoder ML model can be achieved, given that the differences between the augmented versions of the INRs will be more significant than for a different architecture, in which application of such augmentation results in a smaller percentage of weights in the INR being changed. In other examples, the INRs may comprise Sinusoidal Representation Networks (SIRENs), and/or Rectified Fourier Feature Networks (RFFNets).

The medical images represented by the obtained INRs may comprise a wide range of different examples of medical images. In some examples, the medical images may comprise at least one of: Computed Tomography (CT) images, Cone Beam CT (CBCT) images, and/or Magnetic Resonance Images.

The method then comprises, for each of the plurality of INRs, repeating, at least twice, the steps of (i) identifying an augmentation for application to the INR, (ii), applying the identified augmentation to the INR, and (iii) inputting the augmented version of the INR to an encoder ML model, wherein the encoder ML model is operable to output a vector representation of the augmented version of the INR, and wherein the vector representation is of a lower dimension than the INR.

In some examples, for each of the plurality of INRs, identifying an augmentation for application to the INR may comprise identifying an augmentation that differs from an augmentation that has already been applied to the INR in the current iteration of the method. In some examples, this may comprise identifying an augmentation that differs in at least one of: augmentation type, and/or augmentation intensity, from an augmentation that has already been applied to the INR in the current iteration of the method. By using different augmentations (that is different types of augmentation or different intensities of the same type of augmentation), the encoder model is presented with a range of (two or more) different augmented versions of each INR, and so is trained to prioritize those parts of the INR that contain the most important information for recreating the represented image; these are the parts that remain constant across different augmented versions.

According to different examples of the present disclosure, identifying an augmentation for application to the INR may comprise identifying at least one of the following options:
(i) dropout of network weights,
(ii) addition of gaussian noise to network weights and/or biases,
(iii) when using Rectified Linear Unit (ReLU) activations, scaling weights of one layer by a factor “a”, and scaling the weights of the subsequent layer by 1/“a”,
(iv) modifying weights of the first layer of the network to simulate translation, rotation and/or scaling of the input,
(v) permuting the neurons at any layer.

In some examples, identifying an augmentation for application to the INR may comprise randomly selecting an augmentation that differs from an augmentation that has already been applied to the INR in the current iteration of the method. The random selection may be applied for example only to a single aspect in which the augmentation differs from a previously selected augmentation in the current iteration, or may be applied to multiple aspects. For example, in one implementation, a single augmentation type may be selected, and then for each application of that augmentation type to an INR, an intensity of the augmentation may be randomly selected such that the intensity is different to an intensity of the augmentation that has already been applied to that INR in a current iteration of the method.

Following application of the augmentations to the obtained INRs, the method then comprises determining a pairwise similarity between pairs of vector representations output by the encoder ML model. In some examples the method may comprise determining a pairwise similarity between all possible pairs of vector representations output by the encoder ML model.

The method then comprises updating trainable parameters of the encoder ML model to minimize a loss function, wherein the loss function rewards increased similarity between vector representations of augmented versions of the same INR. The loss function may for example output a value that reduces with increasing similarity between vector representations of augmented versions of the same INR. It will be appreciated that in some examples the loss function may be recast as a reward function to be maximized, and thus the reference to minimizing the loss function may be understood as a step that involves optimizing the relevant function with respect to a target direction.

In some examples of the present disclosure, the loss function may additionally penalize similarity between vector representations of augmented version of different INRs. In this manner, the method may learn to ensure that clusters corresponding to different INRs are separated with clear boundaries between them in the latent space of the encoder, so ensuring that the lower dimensional representations, in addition to maintaining the most important information from the INRs, provide diverse input and/or training data for a downstream learning task.

According to some examples of the present disclosure, the loss of the loss function may be proportional to similarity between vector representations of augmented versions of the same INR.

According to some examples of the present disclosure, for a given vector representation of an augmented version of an INR, the loss function may comprise a function of:
(i) a similarity between the given vector representation of an augmented version of an INR, and a vector representation of another augmented version of the same INR, and
(ii) a sum of similarities between the given vector representation of an augmented version of an INR, and vector representations of augmented versions of other INRs in the plurality.

In some examples, the loss function may comprise a normalized, temperature scaled cross-entropy loss.

In further examples, the loss function may comprise:

$${l}_{i , j} = - log \frac{exp ( sim ( {z}_{i} , {z}_{j} ) / τ )}{{\sum }_{k = 1}^{2 N} {[ k \neq i ]}_{} exp ( s i m ( {z}_{i} , {z}_{j} ) / τ )}$$

Where: [symbol] _{i,j} is the loss for a positive pair of examples i and j; a positive pair being a pair of vector representations of augmented versions of the same INR;
zᵢ and zⱼ are the vector representations i and j mapped into a space in which the contrastive loss is applied;
[symbol] _{[k≠i]}∈{0,1} is an indicator function evaluating to 1 if k≠1;
N is the number of INRs; and
τ is a temperature parameter.

According to some examples of the present disclosure, the lower dimensional representations of the plurality of INRs may be suitable for input to a downstream ML task.

In some examples, the method may further comprise iterating the method steps until a convergence criterion is reached, using the encoder ML model to generate vector representations of the plurality of INRs, and outputting vector representations of the plurality of INRs. As discussed above, the convergence criterion may comprise an evaluation of the loss function or its evolution in time and/or with iterations of the method, a number of iterations, a time limit, or any other convergence or termination criterion. Once the convergence criterion is reached, the encoder ML model may be assumed to be sufficiently trained (i.e., the trainable parameter values updated to a point) that the vector representations of INRs output by the encoder ML model will fulfill the desired criteria of having lower dimensionality while maintaining the functional characteristics of the original INR representations. The functional characteristics may be understood as the ability of the INR, or vector representation of the INR, to encode the cognitive content, that is the information contained within, the medical image that is represented.

According to some examples of the present disclosure, the method may further comprise using the output vector representations of the plurality of INRs in an ML task. The ML task may comprise at least one of a modeling task, a generative modeling task, a classification task, a regression task, a clustering task, and/or an anomaly detection task. In some examples, the lower dimensional representations of the plurality of INRs may be for use in at least one of, training, testing, or validating an ML model for an ML task and/or inputting to an ML model that has been trained to perform an ML task.

Another example computer implemented method is presented herein for generating a lower dimensional representation of an INR of a medical image, wherein the representation is for use in an ML task. As for the methods presented above, the method presented below may be performed by a processing node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualized function, or any other logical entity. The processing node may encompass multiple logical entities, as discussed in greater detail below.

The method for generating a lower dimensional representation of an INR of a medical image comprises obtaining an INR representing a medical image, and inputting the INR to an encoder ML model. According to the method, the encoder ML model is operable to output a vector representation of the INR, the vector representation being of a lower dimension than the INR. Also according to the method, the encoder ML model has been trained using a method according to the present disclosure.

The present disclosure also provides a computer program product comprising a computer readable medium, the computer readable medium having computer readable code embodied therein, the computer readable code being configured such that, on execution by a suitable computer or processor, the computer or processor is caused to perform a method according to the present disclosure.

Examples of the present disclosure enable the generation of lower dimensional representations of INRs of medical images. Such lower dimensional representations may be more suitable for processing by existing ML models, frameworks and architectures than the INRs that the represent. The medical images represented by the INRs may for example comprise CT or CBCT images, or MRI images. An important use for CT and CBCT images, in one example, is in the planning and delivery of Radiotherapy, which may be used to treat cancers or other conditions in human or animal tissue. The treatment planning procedure for radiotherapy may include using a three dimensional image of the patient to identify a target region, for example the tumour, and to identify organs near the tumour, termed Organs at Risk (OARs). A treatment plan aims to ensure delivery of a required dose of radiation to the tumour, while minimising the risk to nearby OARs. A treatment plan for a patient may be generated in an offline manner, using medical images that have been obtained using, for example classical CT. These images are generally referred to in this context as diagnostic or planning CT images. The radiation treatment plan includes parameters specifying the direction, cross sectional shape, and intensity of each radiation beam to be applied to the patient. The radiation treatment plan may include dose fractioning, in which a sequence of radiation treatments is provided over a predetermined period of time, with each treatment delivering a specified fraction of the total prescribed dose. Multiple patient images may be required during the course of radiotherapy treatment, and owing to their speed, convenience, and lower cost, CBCT images, as opposed to classical CT images, may be used to determine changes in patient anatomy between delivery of individual dose fractions.

Analysis of CT and CBCT images for the development and delivery of a radiotherapy treatment plan has been enhanced with Machine Learning, with the aim of improving accuracy and repeatability, and reducing the clinician time required for this process. Analysis tasks for which ML techniques have been explored include image reconstruction, scatter, noise and artifact reduction, image segmentation, etc. Performing such ML tasks on Neural Datasets as opposed to standard arrays representing the CT or CBCT scans, can offer particular advantages, as discussed below.

According to existing techniques for performing ML tasks on CT or CBCT images, it is first necessary to use traditional reconstruction methods in order to generate reconstructed images from the measurement data captured in the 2D projections of a patient. These reconstructed images are then used as input to the ML model for performing the downstream ML task, such as segmentation of a target tumor and nearby organs at risk. Traditional reconstruction methods address the inverse problem of obtaining a reconstructed patient volume from the measured intensity values present in projection data. In contrast, when fitting an INR to a medical image, the process of fitting the INR effectively models the data acquisition process, i.e., the INR models the process by which X-rays are attenuated by the patient volume, with this modeling being supervised by the obtained measurements. Medical images encoded with INRs ae thus inherently more explicitly representative of the underlying patent volume than arrays representing a reconstructed image, in addition to being able to handle data sampled at different resolutions. It may consequently be inferred that downstream ML tasks performed on the more explicit representation of the patient volume that is provided by INRs will result in improved performance.

A challenge in the use of INRs of ML tasks as discussed above is that existing architectures for such tasks may struggle to interpret the high dimensionality of INR data. Lower dimensional representations of INRs generated according to the present disclosure may maintain the important information contained within the INRs, so preserving the advantages of using INRs for ML tasks, while also being more suited to processing by existing architectures for such tasks.

As discussed above, the methods presented herein may be performed by a processing node, and the present disclosure provides a processing node that is adapted to perform any or all of the steps of the above discussed methods. The processing node may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The processing node may encompass multiple logical entities, as discussed in greater detail below.

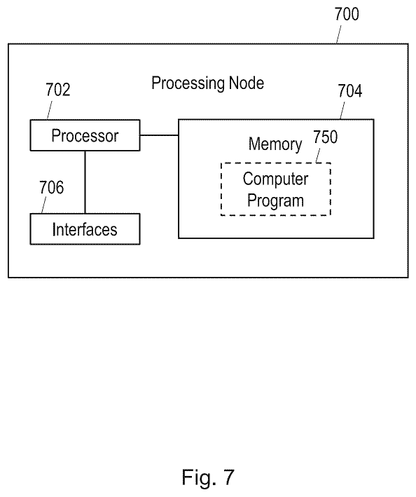

FIG. 7 illustrates one example processing node 700, which may implement the methods disclosed herein as discussed above, for example on receipt of suitable instructions from a computer program. The example processing node 700 comprises a processor or processing circuitry 702, and may comprise a memory 704 and interfaces 706. The processing circuitry 702 is operable to perform some or all of the steps of the methods disclosed herein. The memory 704 may contain instructions executable by the processing circuitry such that the example processing node is operable to perform some or all of the steps of the methods disclosed herein. The instructions may also include instructions for executing one or more telecommunications and/or data communications protocols. The instructions may be stored in the form of the computer program 750. In some examples, the processor 702 or processing circuitry may include one or more microprocessors or microcontrollers, as well as other digital hardware, which may include digital signal processors (DSPs), special-purpose digital logic, etc. The processor 702 or processing circuitry may be implemented by any type of integrated circuit, such as an Application Specific Integrated Circuit (ASIC), Field Programmable Gate Array (FPGA) etc. The memory may include one or several types of memory suitable for the processor, such as read-only memory (ROM), random-access memory, cache memory, flash memory devices, optical storage devices, solid state disk, hard disk drive, etc.

In some examples as discussed above, the example processing node 700 may be incorporated into treatment apparatus, and examples of the present disclosure also provide a radiotherapy treatment apparatus comprising either or both of a processing node as discussed above and/or a planning node operable to implement a method for adapting a radiotherapy treatment plan.

The above discussion provides an overview of methods which may be performed according to different examples of the present disclosure. These methods may be performed by a processing node.

There now follows a detailed discussion of how different process steps illustrated and discussed above may be implemented. The functionality and implementation detail described below is discussed with reference to the modules of the processing nodes performing the methods substantially as described above.

In the following example implementation, we focus on the widely acclaimed SimCLR algorithm (Chen et al., 2020). SimCLR is a contrastive learning framework for learning visual representations. At its core, it learns by maximizing the alignment of the representations of augmented views of the same image. The structure of SimCLR is described in below. The following discussion of implementation of the methods disclosed herein with SimCLR offers the following contributions:

We show how contrastive learning can be applied to implicit neural representations with different architectures.

We characterize the permutation symmetries of multiplicative filter networks and provide further evidence for the importance of processing weights with functions that are invariant with respect to the permutation symmetries of the INRs.

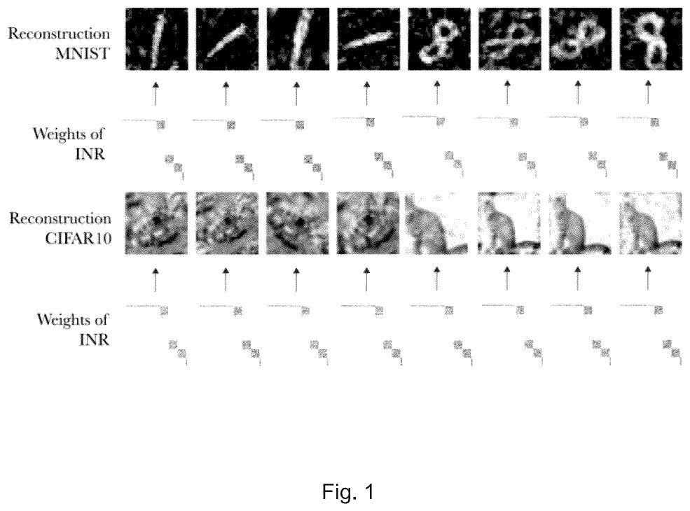

FIG. 1 is a representation of the example implementation discussed herein, applied to publicly available datasets. We train an encoder with SimCLR on implicit neural representations (INR). To obtain the pairs at the core of the method we augment the weights of the INR. These are standard augmentations such as random drop-out and Gaussian noise, and geometric augmentations such as rotations, translations, and scaling. The latter are constructed to obtain the desired transformation on the reconstructed image, while changing only the weights of the INR. Here we show the edge feature matrix of a multiplicative filter network (MFN), which also shows the connectivity of the computation graph of the neural field. This is what is ultimately used in the encoder architecture (Zhang et al., 2023). Each square block corresponds to a hidden layer in the network, the horizontal line at the top is the weights of filters, while the diagonal block shows the element-wise products used in an MFN between filters and hidden layers.

### Datasets of INRs

A dataset of implicit representation is simply a set {θᵢ} of neural field parameters, each one reconstructing an image in a dataset of images. To build a dataset of implicit neural representations, several design choices have to be made, such as the network architecture and the reconstruction accuracy to name a few. We therefore experimented with a combination of these design choices as reported in Table 1. An important feature of our datasets is the number of implicit representations that have been obtained per image. For a given signal and a given neural field architecture, there exist multiple functionally equivalent implicit neural representations. This is due to the existence of multiple local minima of the optimization problem of Equation 2. As described in the following sections, the proposed method is aligned with the implicit working hypotheses of recent works (Navon et al., 2023; Zhang et al., 2023: namely that permutation symmetries of the neural fields parameterizations account for most of them. Our experiments provide further evidence for the validity of this hypothesis.

### Neural Field Architectures

For our experiments we use datasets of implicit representations constructed using SIRENs (Sitzmann et al., 2020) and Multiplicative Filters Networks (Fathony et al., 2020). Despite SIRENs being the most popular INR architecture in the literature, in the discussion below we explain how multiplicative filters networks are more amenable to augmentations. We therefore present them as good candidate for contrastive learning.

SIREN (sinusoidal representation networks) Neural Fields (Sitzmann et al., 2020) are a specialized form of multilayer perceptron that have proven to be particularly adept at representing complex functions, including natural images and signals. They are distinguished by their sinusoidal activation functions. SIRENs utilize periodic activation functions to improve the network's capacity to capture variations in data, especially when dealing with wave-based signals and images. The structure of a SIREN network is formalized as:

$${h}^{( 1 )} = x \qquad Equation 3$$

$${h}^{( i )} = sin ( {Ω}_{0} {W}^{( i - 1 )} {h}^{( i - 1 )} + {b}^{( i - 1 )} ) , i = 1 , … , k - 1$$

$${f}_{θ} ( x ) = {W}^{( f )} {h}^{( k )} + {b}^{( k )}$$

where W⁽ⁱ⁻¹⁾∈ [symbol] ^{d}^{ᵢ}^{×d}^{_{i−1}} and b⁽ⁱ⁻¹⁾∈ [symbol] ^{d}^{ᵢ} denote the weight matrix and bias vector for the i-th layer, respectively, and Ω₀ is a scalar. Here, the sin function is utilized as the activation function at each layer. Their capacity to represent and reconstruct intricate data patterns is attributed to the sinusoidal activations, enabling the network to capture a wide range of frequencies and amplitudes effectively. This feature makes SIREN particularly beneficial for tasks involving the modeling and generation of natural signals and images.

Multiplicative Filters Networks (MFNs) (Fathony et al., 2020) are neural fields architectures that unlike feedforward neural networks do not rely on compositional depth for reconstruction power. Instead, MFNs apply nonlinear filters to the input and iteratively multiply together linear functions of those filters. Explicitly, an MFN is defined by the recursion:

$${z}^{( 1 )} = g ( x ; {ψ}^{( 1 )} ) \qquad Equation 4$$

$${z}^{( i + 1 )} = ( {W}^{( i )} {z}^{( i )} + {b}^{( i )} ) ⊙ g ( x ; {ψ}^{( i + 1 )} ) , i = 1 , … , k - 2$$

$${f}_{θ} ( x ) = {W}^{( k - 1 )} {z}^{( k - 1 )} + {b}^{( k - 1 )}$$

where ⊙ represents element-wise multiplication, W⁽ⁱ⁾∈ [symbol] ^{d}^{ᵢ₊₁}^{×d}^{ᵢ}, b⁽ⁱ⁾∈ [symbol] ^{d}^{ᵢ₊₁} and g: R^{d} [symbol] [symbol] ^{d}^{ᵢ} are the nonlinear filters parameterized by ψᵢ that are applied to the input directly. Among the possible choices of the filters, throughout the paper, we use a linear layer composed with a sine function, g(x; ψ⁽ⁱ⁺¹⁾)=sin ωx+ϕ where ωᵢ∈ [symbol] ^{d×d}^{₁} and ϕᵢ∈ [symbol] ^{d}^{₁}. In particular, in their paper, Fathony et al. (2020) prove that such a multiplicative filter network is ultimately just a linear function of an exponential (in k) number of Fourier basis functions.

### Permutations Symmetries

It has been well known (Hecht-Nielsen, 1990) that the parameter space of neural networks is characterized by a combinatorial number of permutation symmetries. In particular, consider any layer of a MLP W⁽ⁱ⁺¹⁾σ(W⁽ⁱ⁾z⁽ⁱ⁾+b⁽ⁱ⁾) and permute the weight matrices and the bias vector as W⁽ⁱ⁾, b⁽ⁱ⁾ [symbol] P^{T}W⁽ⁱ⁾, P^{T}b⁽ⁱ⁾, and W⁽ⁱ⁺¹⁾ [symbol] W⁽ⁱ⁺¹⁾P. The result is a different implicit representation that nonetheless represents the exact same function. In the literature, permutation symmetries of neural networks have been studied from several different perspectives . . . (Chen et al., 1993; Ainsworth et al., 2022; Simsek et al., 2021; Entezari et al., 2021).

Similarly to MLPs, we can characterize the permutation symmetries of MFNs. It is easy to see from Equation 4 that for a MFN parameterized by W⁽ⁱ⁾, b⁽ⁱ⁾, ω⁽ⁱ⁾ and ϕ⁽ⁱ⁾, any set of k−1 permutations (P₁, . . . , P_{M-1}) acting of the weight space as:

$${W}^{( i )} ↦ {P}_{i + 1} {W}^{( i )} {P}_{i}^{T} ; {b}^{( i )} ↦ {P}_{i + 1} {b}^{( i )} 1 \leq i \leq k - 2 \qquad Equation 5$$

$${ω}^{( i )} ↦ {P}_{i} {ω}^{( i )} ; {ϕ}^{( i )} ↦ {P}_{i} {ϕ}^{( i )} 1 \leq i \leq k - 1$$

$${W}^{( k - 1 )} ↦ {W}^{( k - 1 )} {P}_{k - 1}^{T} ; {b}^{( k - 1 )} ↦ {b}^{( k - 1 )}$$

defines a symmetry.

In light of recent findings in the literature, and the results of the experiments presented in this paper, we make the educated hypothesis that permutation symmetries are responsible for a good amount of the impracticability of downstream tasks. This stems from the observation that, once permutation symmetries become irrelevant due to the permutation invariance of encoder, the latter can easily align (high top-k validation accuracy) vector representations of differently initialized INRs. It is important to note that this hypothesis is speculative and not a formal statement, serving as a foundation for further investigation and discussions rather than a conclusive assertion. It is informed by current insights and aims to stimulate further research and exploration into this intricate area.

### Implementation of Methods

In this section we outline the details of the particular implementation of the methods herein. We start from a brief description of the SimCLR framework. It should be noted that the overview we provide is a concise summary and not exhaustive. For a comprehensive understanding and detailed insights on the SimCLR algorithm, readers are encouraged to refer to the original paper (Chen et al., 2020).

### SimCLR

SimCLR (Contrastive Learning of Visual Representations) is a self-supervised learning algorithm introduced for the efficient learning of visual representations. It operates by maximizing the similarity between augmented views of the same data instance while minimizing the similarity between augmented views of different instances. In particular, the SimCLR architecture consists of an encoder f(⋅) and a small MLP projector head g(⋅). SimCLR starts by randomly sampling a minibatch of N examples and generating two distinct augmented views (positive pairs) \tilde{x}ᵢ and \tilde{x}ⱼ for every example. All the augmented views in the minibatch are passed through the encoder and the projector to get zᵢ, zⱼ. The objective of SimCLR is defined by the contrastive loss function, typically the Noise Contrastive Estimation (NCE) loss or the Normalized Temperature-Scaled Cross Entropy Loss (NT-Xent). It is formulated, for a positive pair of examples (i, j) as:

$$l ( i , j ) = - log ( \frac{exp ( s i m ( {z}_{i} , {z}_{j} ) / τ )}{{\sum }_{k = 1}^{2 N} {[ i \neq k ]}_{} exp ( sim ( {z}_{i} , {z}_{j} ) / τ )} ) \qquad Equation 6$$

$$where$$

$$sim ( {z}_{i} , {z}_{j} ) = \frac{{z}_{i} \cdot {z}_{j}}{‖ {z}_{i} ‖ ‖ {z}_{j} ‖}$$

is the cosine similarity between vectors zᵢ and zⱼ, [symbol] _{i≠k}∈{0, 1} is the indicator function, and τ is a temperature parameter that scales the similarities. Intuitively, the contrastive learning task aims to identify \tilde{x}ⱼ in {\tilde{x}ₖ}_{k≠i} for a given \tilde{x}ᵢ. Once the contrastive objective has been optimized, the head projector g(⋅) is thrown away and the encoder f(⋅) is used to get the representations to be used for downstream tasks.

### Data Augmentations

As seen, SimCLR and other contrastive self-supervised learning methods such as MoCo (Momentum Contrast for Unsupervised Visual Representation Learning) (He et al., 2020), and BYOL (Boot-strap Your Own Latent) (Grill et al., 2020), relies on augmentations to systematically define the contrastive prediction task. For discrete pixel representations of images, common augmentations can be broadly categorized into two types based on the nature of the transformation applied to the image. The first category encompasses spatial or geometric transformations, which involve altering the structural form of the data. Examples of these transformations include cropping and resizing often accompanied by horizontal flipping, rotation, as noted by (Gidais at al., 2018), and cutout (DeVries & Taylor, 2017). The second category is characterized by appearance transformations that primarily focus on altering the visual aesthetics of the image without changing its structural integrity. Such augmentations include color distortions like color dropping, and adjustments to brightness, contrast, saturation, and hue, as explored by Howard (2013) and (Szegedy et al., 2015). Additionally, other transformations like Gaussian blur and Sobel filtering fall under this category of augmentations.

Particularly when employed for the type of tasks considered in this work, namely classification, augmentations are transformation of datapoints that preserve their object identity. It is not straightforward to perform any systematic transformation on implicit neural representations in such a way that the object identity of the image they represent is preserved. In other words, it is easy to destroy any semantic information contained in a neural field by acting on its parameters. Here we show how augmentations can be performed on implicit representations to enable contrastive learning. We divide the augmentations into three categories: standard, geometric and random seed augmentations.

Standard augmentations: With standard augmentations we refer to those transformations performed on the datapoints that are commonly used in machine learning to randomly alter the dataset and add some regularization effect to the training. In this work we use Gaussian noise and random drop-out.

Geometric augmentations: With geometric augmentations we refer to the action of certain groups of transformations on the functions that the implicit representations define. Formally, let G be a group of transformations such as the group of rotations or the translation group, and let f_{θ}: [symbol] ²→ [symbol] ³ be a neural field representing an image, as standard practice, we define the group action of g∈G on the set of functions as:

$${L}_{q} {f}_{θ} ( x ) = [ f ∘ {g}^{- 1} ] ( x ) = f ( {g}^{- 1} x ) \qquad Equation 7$$

Operationally, this means that the value of the g-transformed function L_{g}f_{θ}(x) at the point x, is the value of the original function f at the point g⁻¹x, which is the unique point mapped to x by g. For example. At this point, to define the augmentation t_{g}:θ [symbol] t(θ) we need to find a transformation of the weights θ such that:

$${f}_{{t}_{g} ( θ )} ( x ) = {L}_{g} {f}_{θ} ( x ) = [ f ∘ {g}^{- 1} ] ( x ) = f ( {g}^{- 1} x ) ∀ x ∈ {ℝ}^{2} \qquad Equation 8$$

For transformations such as rotations and scaling, their group action on [symbol] ² is simply a matrix multiplication, i.e., for every g, g⁻¹x=R_{g}x for some R∈ [symbol] ²×2. It is straightforward to note that, for MLP, the action of t_{g} on θ simply consists of multiplying from the right by R_{g} the first weight matrix. In the case of MFNs, it consists of multiplying from the right by R_{g} every filter matrix. For translations, t_{g} does not affect weight matrices but acts on the biases of the first layer in the case of MLPs as t_{g}(b⁽¹⁾)=b⁽¹⁾−W⁽¹⁾t and all the biases in the filter layers in the case of MFNs as:

$${t}_{g} ( {ϕ}^{( i )} ) = {ϕ}^{( i )} - {ω}^{( i )} t , i - 1 , … , k - 1$$

At this point it is worth noting that for different architectures a different proportion of parameters is affected by augmentations. In general, for contrastive learning to extract the relevant features from a dataset of INRs, the more these are affected by augmentations, the better. For MFNs the proportion of weights affected by geometric augmentations is considerably higher than MLPs. For example, geometric augmentations on MLPs alter is:

$$\frac{3}{d \times ( n - 1 ) + 3}$$

whereas in an MFN with n layers and hidden dimension d the proportion of weights altered by geometric augmentations is:

$$\frac{1}{( d + 1 ) + \frac{1}{n}}$$

For example, for an MFN with 4 layers and hidden dimension 4 such proportion is about 0.19 which is considerably higher than that of a 2 hidden layer MLP with hidden dimension 32 which is about 0.09.

Random seed augmentations Fitting a single image from different initializations results in different INRs. Our datasets are therefore made of multiple INRs for every image, obtained from different initialization. During training, two positive pairs are always obtained augmenting different INRs obtained starting from different initializations.

### Aliasing

It can happen that two completely different INRs are indistinguishable when sampled at discrete locations, because of aliasing. A basic result in signal processing is given by the Nyquist-Shannon sampling theorem. This states that to sample a finite band signal without loss of information, it must be sampled with a frequency at least double the frequency of the spectral component of the information signal at a higher frequency (also called Nyquist frequency). It is easy to compute the highest frequency of an MFN: it is the sum of the maximum frequencies of filters, which ultimately are the absolute values of entries of the filter matrices. We therefore propose to add the following regularizer to the reconstruction loss Equation 2:

$$ℛ ( θ ) = {\sum }_{i = 1}^{k - 1} {\vert {ω}_{i} \vert}_{0} \qquad Equation 9$$

where |⋅|₀ is the L₀ norm.

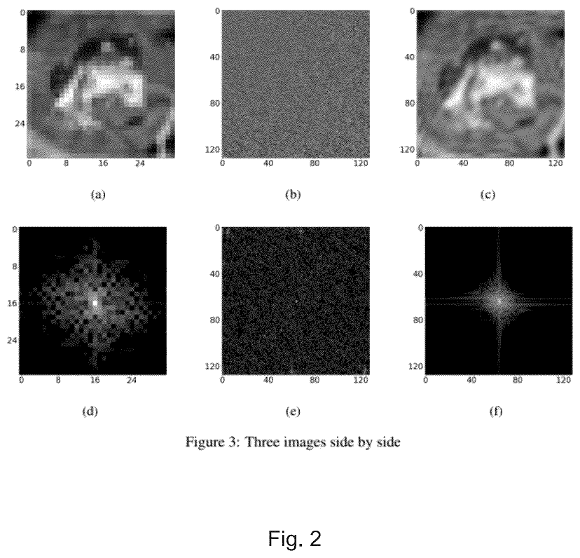

As reported in Fathony et al. (2020) initializations are crucial to get good reconstruction accuracy. Empirically, we find that initializations are also key to avoiding aliasing. Our regularizer obviates the need to find a trade-off between good reconstruction and aliasing, by allowing us to initialize the MFN with higher frequencies around the Nyquist, and keeping them below while fitting. FIG. 2 shows the effects of the proposed regularization.

FIG. 2 illustrates aliasing in multiplicative filters networks. (a) An MFN trained without anti-aliasing regularization sampled on the training grid. (b) The same MFN sampled on a finer grid. (c) An MFN trained with anti-aliasing regularization. (d) The frequency spectrum of the original image. (e) The frequency spectrum computed with a higher spatial resolution. Outside the box frequencies should be zero. (f) The frequency spectrum of an MFN trained with anti-aliasing regularization.

### Encoder Network Architecture

Our encoder network is based on the work of Zhang et al. (2023). In this work, the authors propose to use the computational graph of neural networks and encode INRs with graph networks or transformers that respect the permutation symmetries present in the parameter space. Under the computational graph paradigm, the biases of each layer correspond to node features, while the weights of each layer correspond to edge features. For a standard fully-connected MLP, the edge features matrix is organized as a block-superdiagonal matrix, i.e. a block matrix with blocks populated 1 above and to the right of the main diagonal (see FIG. 1). The authors extend recent graph network and transformer architectures, namely PNA (Corso et al., 2020), and Relational Transformer (Diao & Loynd, 2023) to better accommodate edge features since the bulk of the information in the computational graph is in the edge features.

### Experiments

We evaluate our method on two datasets CIFAR10 and MNIST, and compare it to a supervised learning method that uses the same architecture that we use in the encoder in SimCLR.

### Dataset

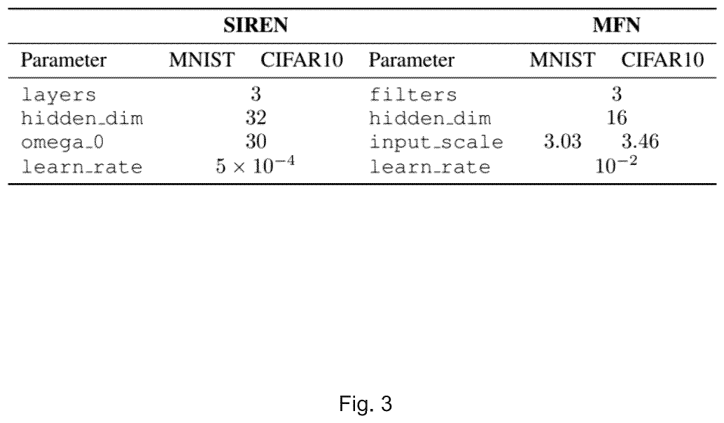

We train our model using four INR datasets. Of these, two of them are obtained by fitting on MNIST, while the other two by fitting on CIFAR10. For both datasets, 30 INRs are trained per image and used for random seed augmentations. FIG. 3 shows Table 1, which illustrates hyperparameters used in the datasets of Implicit Neural Representations.

### Experimental Setup

We use the Relational Transformer architecture from Zhang et al. (2023) without probe features for both contrastive learning and supervised learning experiments. Essentially, probe features are the activations of every layer, including the output layer, obtained using learnable inputs. We chose not to use those in our experiments, to show that our method learns in weight space and does not require querying the neural field to perform well. The architecture is the same for all experiments. The optimizer is Adam (Kingma & Ba, 2014) with different learning rates for each experiment. We noticed that the learning rate had a great impact on the ability of the model to fit the data.

When performing augmentations for contrastive learning we first load a batch of two random seeds of INRs fit to the same images. Then, we apply random augmentations from the set described in the previous section to each INR.

### Results

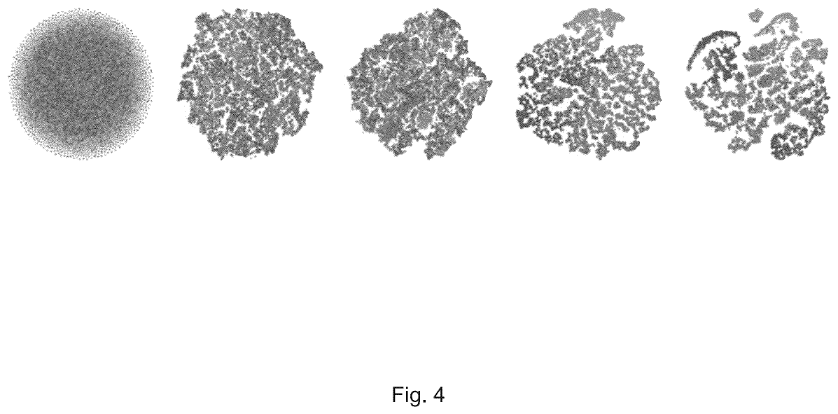

We first looked at the embeddings obtained using the learned encoders and compared them to the weights of the INRs using t-SNE Van der Maaten & Hinton (2008). The contrastive method successfully results in structured embeddings in MNIST, as seen in FIG. 4. We found the model struggling with CIFAR10 for both SIREN and MFN, because of the increased complexity of the data.

FIG. 4 illustrates embeddings of the datasets using t-SNE. On the left, we used the weights of the INRs without any processing, no structure is present and they are distributed seemingly at random. Follow the MFN CIFAR10 and SIREN CIFAR10 (in this order). This dataset is harder to fit, however, some structure emerges. Finally, MFN MNIST and SIREN MNIST. Different colors represent different class labels.

We then measured the accuracy of the supervised method on the validation set. For the contrastive methods, we fit a linear probe on the embeddings predicted using the frozen encoder on the training set. The contrastive method surprisingly performs more closely to the supervised one on the MFN, compared to SIREN. This suggests that performing contrastive learning is more beneficial for certain architectures of neural fields. Further work is needed to investigate what architectures self-supervised learning performs better.

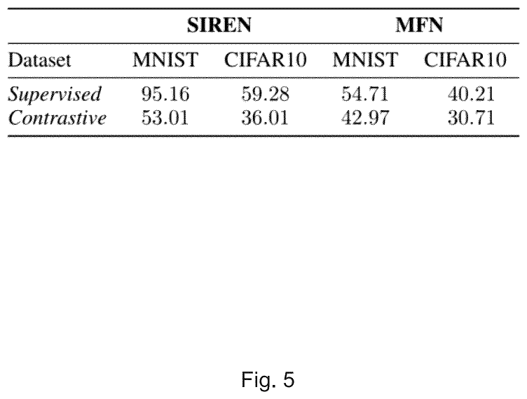

FIG. 5 shows Table 2, which illustrates accuracy (%) for all INRs used, with both the supervised method and a linear probe on the features obtained using the frozen encoder. We used the best accuracy across different runs for all models.

### Related Work

Implicit Neural Representations The design of deep learning architectures to process the parameters of neural networks is a relatively new research direction. Here we provide an overview of the most relevant pioneering studies in these field.

The works of Eilertsen et al. (2020) and Unterthiner et al. (2020) are centered around predicting attributes of trained neural networks (NNs) by examining their weights. Eilertsen et al. (2020) focuses on estimating the hyperparameters employed during the network's training phase, while Unterthiner et al. (2020) is dedicated to assessing the network's capacity for generalization. Both investigations involve the application of standard NNs to the flattened weights or their statistics. Xu et al. (2022) introduced a concept wherein NNs are processed through the application of another NN to a combination of their high-order spatial derivatives, a technique particularly suited for implicit neural representations (INRs) where derivative information is pertinent. However, the adaptability of these networks to broader tasks remains ambiguous, and the necessity for high-order derivatives can impose a significant computational load. Dupont et al. (2022) proposed a novel approach to deep learning tasks like generative modeling, applying them to a collection of INRs derived from the initial data. They advocated for the meta-learning of concise vectors, referred to as modulations, which are integrated into a neural network, with parameters consistent across all training instances, to achieve meaningful data representations. In our work, we opted not to use conditioned neural fields nor the meta-learning initialization technique such as the one proposed by Tancik et al. (2021). This is to test how our method performs with out-of-the-shelf implicit representations that can be obtained easily, without the need of the shared-across-networks parameters nor the meta-learned initialization that might not work as a good initialization for different datasets. Finally, relevant works for our method are that of Navon et al. (2023) and Zhou et al. (2023), from which we adapted the proposed transformer-like architecture to work with multiplicative filters networks, and that of Zhang et al. (2023), which first demonstrated the importance of augmentations and permutation invariant architectures for processing weights of neural fields, and that of Navon et al. (2023); Zhou et al. (2023), which first demonstrated the importance of augmentations and permutation invariant architectures for processing weights of neural fields.

Contrastive Learning The field of Self-Supervised Learning (SSL) is rapidly advancing, focusing on utilizing unlabeled visual data. Contemporary strategies primarily depend on comparing embeddings derived from transformed input images. This approach is rooted in the concept of aligning image representations subjected to minor alterations, a notion introduced by Becker and Hinton. In this context, SSL techniques fall into two primary classifications: contrastive learning and non-contrastive learning. This study narrows its exploration to contrastive learning methods such as Moco (He et al., 2020) and BYOL Grill et al. (2020) and in particular to SimCLR Chen et al. (2020). Relevant to our study is the work of Schurholt et al. (2021) where they propose to perform self-supervised learning on the weights of neural networks to predict model characteristics. They differ from us as they do consider INRs and do not use an encoder that is invariant to permutations. Therefore propose to use permutations as augmentations. Finally, Navon et al. (2023) tested their permutation invariant architecture in a simplified contrastive learning setting.

### Final Comments

Implicit Neural Representations have emerged as an interesting alternative to traditional array representations. The challenge of performing downstream tasks directly on implicit representations has been addressed by several methods. Overcoming this challenge would open the door to the application of implicit representations to a wide range of fields. Then again, self-supervised representation learning methods, such as the several contrastive learning frameworks which have been proven powerful representation learning methods. So far, the use of self-supervised learning for implicit representations has remained unexplored, mostly because of the difficulty of producing valid augmented views of implicit representations to be used for learning contrasts. In examples of the present disclosure, the popular SimCLR algorithm is adapted to implicit representations that consist of multiplicative filters networks and SIRENs. While methods to obtain augmentations in SIREN have been studied in the literature, we provide methods for augmenting MFNs effectively. We show how MFNs lend themselves well to geometric augmentations.

The author have demonstrated the applicability and extensive potential of self-supervised learning to implicit neural representations. Our findings spotlight SSL as an interesting research direction in the field of implicit representations, showcasing its ability to effectively learn useful representations from unlabeled datasets of INRs. In that regard, one key finding is the importance of the random seed augmentations, as described above.

We propose MFNs as a candidate INR architecture for the larger proportion of parameters that are affected by geometric augmentations, such as rotations, scaling, and translations. We also provide a method to regularize MFNs and avoid aliasing. Other than obtaining good reconstructions, this method also results in a constrained implicit representation space. We find that fitting regularized INRs results in better downstream performances.

The experiments presented herein provide further evidence for the hypothesis that permutation symmetries represent the most significant challenge in processing the weights of neural networks. This stems from the observation that very expressive architectures fail to align, in terms of top-1 and top-5 validation accuracy, positive pairs obtained with random seed augmentations. Conversely, permutation invariant architectures rapidly achieve good alignment.

To the best of our knowledge, our work is the first to demonstrate that self-supervised learning on implicit representations of images is feasible and results in good downstream task performances.

The methods of the present disclosure may be implemented in hardware, or as software modules running on one or more processors. The methods may also be carried out according to the instructions of a computer program, and the present disclosure also provides a computer readable medium having stored thereon a program for carrying out any of the methods described herein. A computer program embodying the disclosure may be stored on a computer readable medium, or it could, for example, be in the form of a signal such as a downloadable data signal provided from an Internet website, or it could be in any other form.

It should be noted that the above-mentioned examples illustrate rather than limit the disclosure, and that those skilled in the art will be able to design many alternative embodiments without departing from the scope of the appended claims or numbered embodiments. The word “comprising” does not exclude the presence of elements or steps other than those listed in a claim or embodiment, “a” or “an” does not exclude a plurality, and a single processor or other unit may fulfil the functions of several units recited in the claims or numbered embodiments. Any reference signs in the claims or numbered embodiments shall not be construed so as to limit their scope.

What is claimed is:

1. A computer implemented method for training a Machine Learning (ML) model to generate lower dimensional representations of a plurality of Implicit Neural Representations (INR), wherein each INR represents a medical image, the method comprising:
obtaining a plurality of INRs;
for each of the plurality of INRs, repeating, at least twice:
identifying an augmentation for application to the INR;
applying the identified augmentation to the INR to create an augmented version of the INR; and
inputting the augmented version of the INR to an encoder ML model, wherein the encoder ML model is operable to output a vector representation of the augmented version of the INR, and wherein the vector representation is of a lower dimension than the INR;
determining a pairwise similarity between pairs of vector representations output by the encoder ML model; and
updating one or more trainable parameters of the encoder ML model to minimize a loss function, wherein the loss function rewards an increased similarity between vector representations of augmented versions of a same INR.

2. The method as claimed in claim 1, wherein the loss function penalizes a similarity between vector representations of an augmented version of different INRs.

3. The method as claimed in claim 1, wherein, for each of the plurality of INRs, identifying an augmentation for application to the INR comprises identifying an augmentation that differs from an augmentation that has already been applied to the INR in a current iteration of the method.

4. The method as claimed in claim 3, wherein, for each of the plurality of INRs, identifying an augmentation for application to the INR comprises identifying an augmentation that differs in at least one of an augmentation type or an augmentation intensity from an augmentation that has already been applied to the INR in the current iteration of the method.

5. The method as claimed in claim 1, wherein identifying an augmentation for application to the INR comprises identifying at least one of a dropout of network weights or an addition of gaussian noise to at least one of the network weights or network biases, and wherein the method further comprises:
when using a Rectified Linear Unit (ReLU) activation, scaling weights of a first layer by a factor “a”, and scaling the weights of a subsequent layer by 1/“a”;
modifying weights of the first layer of the network to simulate translation, rotation and/or scaling of the input; and
permuting neurons at any layer.

6. The method as claimed in claim 1, wherein identifying an augmentation for application to the INR comprises randomly selecting an augmentation that differs from an augmentation that has already been applied to the INR in a current iteration of the method.

7. The method as claimed in claim 1, wherein the INRs comprise at least one of Multiplicative Filter Networks, Sinusoidal Representation Networks (SIRENs) or Rectified Fourier Feature Networks (RFFNets).

8. The method as claimed in claim 1, wherein the loss of the loss function is proportional to similarity between vector representations of augmented versions of the same INR.

9. The method as claimed in claim 1, wherein, for a given vector representation of an augmented version of an INR, the loss function comprises a function of:
a similarity between the given vector representation of an augmented version of an INR, and a vector representation of another augmented version of the same INR; and
a sum of similarities between the given vector representation of an augmented version of an INR, and vector representations of augmented versions of other INRs in the plurality of INRs.

10. The method as claimed in claim 1, wherein the loss function comprises a normalized, temperature scaled cross-entropy loss.

11. The method as claimed in claim 1, wherein the loss function comprises:
$${l}_{i , j} = - log \frac{exp ( s i m ( {z}_{i} , {z}_{j} ) / τ )}{{\sum }_{k = 1}^{2 N} {[ k \neq i ]}_{} exp ( s i m ( {z}_{i} , {z}_{j} ) / τ )} .$$

12. The method as claimed in claim 1, wherein the lower dimensional representations of the plurality of INRs are input to a downstream ML task.

13. The method as claimed in claim 12, further comprising:
iterating operations of the method until a convergence criterion is reached;
using the encoder ML model to generate vector representations of the plurality of INRs, and
outputting vector representations of the plurality of INRs.

14. The method as claimed in claim 13, further comprising:
using the output vector representations of the plurality of INRs in an ML task.

15. A method as claimed in claim 12, wherein the ML task comprises at least one of a modeling task, a generative modeling task, a classification task, a regression task, a clustering task, or an anomaly detection task.

16. The method as claimed in claim 1, wherein the lower dimensional representations of the plurality of INRs are for use in at least one of, training, testing, or validating a ML model for an ML task, and wherein the method further comprises:
inputting to an ML model that has been trained to perform an ML task.

17. A computer implemented method for generating lower dimensional representation of an Implicit Neural Representation (INR) of a medical image, wherein the representation is for use in a Machine Learning (ML) task, the method comprising:
obtaining an INR representing a medical image; and
inputting the INR to an encoder ML mode, wherein the encoder ML model is operable to output a vector representation of the INR, the vector representation being of a lower dimension than the INR, and wherein the encoder ML model is trained by:
obtaining a plurality of INRs;
for each of the plurality of INRs, repeating, at least twice:
identifying an augmentation for application to the INR;
applying the identified augmentation to the INR to create an augmented version of the INR; and
inputting the augmented version of the INR to an encoder ML model, wherein the encoder ML model is operable to output a vector representation of the augmented version of the INR, and wherein the vector representation is of a lower dimension than the INR;
determining a pairwise similarity between pairs of vector representations output by the encoder ML model; and
updating one or more trainable parameters of the encoder ML model to minimize a loss function, wherein the loss function rewards an increased similarity between vector representations of augmented versions of a same INR.

18. The method as claimed in claim 17, wherein the medical images comprise at least one of a computed tomography (CT) image, a cone beam CT (CBCT) image, or a magnetic resonance image.

19. A processing node for training a Machine Learning (ML) model to generate lower dimensional representations of a plurality of Implicit Neural Representations (INR), each INR representing a medical image, the processing node comprising processing circuitry configured to cause the processing node to:
obtain a plurality of INRs;
for each of the plurality of INRs, repeat, at least twice:
identify an augmentation for application to the INR;
apply the identified augmentation to the INR to create an augmented version of the INR; and
input the augmented version of the INR to an encoder ML model, wherein the encoder ML model is operable to output a vector representation of the augmented version of the INR, and wherein the vector representation is of a lower dimension than the INR;
determine a pairwise similarity between pairs of vector representations output by the encoder ML model; and
update one or more trainable parameters of the encoder ML model to minimize a loss function, wherein the loss function is configured to reward increased a similarity between vector representations of augmented versions of a same INR.

20. The processing node as claimed in claim 19, wherein the processing circuitry is further configured to:
cause the processing node to penalize a similarity between vector representations of an augmented version of different INRs.

21. The processing node of claim 20, wherein the processing node is included in a radiotherapy treatment apparatus.
